(19) United States
(12) Patent Application Publication
(10) Pub. No.: US 2025/0174329 A1
(43) Pub. Date: May 29, 2025

(54) TOUCH-FREE MEDICAL TREATMENT

(71) Applicant: Guided Healthcare Inc., Phoenix, AZ (US)

(72) Inventors: John Ellis Stafira, Dallas, TX (US); Jeremy William Crotts, Phoenix, AZ (US); Joseph Alexander Dallal, Newport Beach, CA (US); Amy Summers, Laguna Niguel, CA (US)

(21) Appl. No.: 18/841,661

(22) PCT Filed: Feb. 27, 2023
(86) PCT No.: PCT/US2023/013933

§ 371 Date: Aug. 26, 2024

Related U.S. Application Data
(60) Provisional application No. 63/314,625, filed on Feb. 28, 2022.

(51) Int. Cl. G16H 20/10; G16H 10/40

(52) U.S. Cl. CPC G16H 20/10; G16H 10/40

(57) ABSTRACT

A method for touch-free medical treatment of a patient includes identifying a prospect as the candidate and providing the candidate with a lab test based on identifying the prospect as the candidate. The method also includes obtaining lab results from applying the lab test to a biometric sample from the candidate. The method further includes determining whether the candidate is suitable for treatment as a patient therapy based on the lab results. When the candidate is suitable for treatment, the method includes reviewing the lab test, patient medical history, and demographic characteristics of the candidate in a review. The method also includes titrating a level of a customized drug or other therapy for the patient based on the review; and prescribing the customized drug or other therapy for the patient at the titrated level based on the review.

### CROSS REFERENCE TO RELATED APPLICATIONS

This United States non-provisional patent application claims priority to U.S. provisional patent application No. 63/314,625, filed Feb. 28, 2022, which is hereby incorporated by reference in the entirety.

### BACKGROUND

### 1. Field of the Disclosure

The present disclosure relates to the field of medical treatments. More particularly, the present disclosure relates to touch-free medical treatments.

### 2. Background Information

Costs and complexity of finding and obtaining medical care have recently come into focus in places like the United States. Recently, the occasional importance of reducing physical contact between persons has also come into focus due, for example, to the worldwide Coronavirus-19 pandemic.

The current healthcare system is impacted by errors and omissions that can have significant effects on patient outcomes. Additionally, many medical practices rely on anecdotal evidence rather than empirical evidence from their entire patient population when making treatment decisions.

The sharing of health data is subject to barriers in traditional healthcare systems, and this can result in data being siloed off and not used to its full potential. Examples of these barriers include technical challenges, privacy and security concerns, legal and regulatory issues, financial incentives, and culture.

The current model of digital health in medicine lacks continuity and a standard of care, which can lead to a variety of patient outcomes in terms of clinical efficacy and patient satisfaction. As a result, patients may experience mixed quality outcomes, which can negatively impact their overall healthcare experience.

### BRIEF DESCRIPTION OF THE DRAWINGS

FIG. 1 illustrates a method for touch-free medical treatment, according to an aspect of the present disclosure;

FIG. 2 illustrates a networked arrangement for touch-free medical treatment, according to an aspect of the present disclosure;

FIG. 3 illustrates a flow of movement for a lab test in touch-free medical treatment, according to an aspect of the present disclosure;

FIG. 4 illustrates a method of performing a laboratory analysis from a lab test in touch-free medical treatment, according to an aspect of the present disclosure;

FIG. 5 illustrates a method of obtaining lab results from a laboratory analysis in touch-free medical treatment, according to an aspect of the present disclosure;

FIG. 6 illustrates a method of identifying a suitable candidate from lab results in touch-free medical treatment, according to an aspect of the present disclosure;

FIG. 7 illustrates a method of conducting a telemedicine session in touch-free medical treatment, according to an aspect of the present disclosure;

FIG. 8 illustrates a method of titrating a level of a customized drug or other therapy in touch-free medical treatment, according to an aspect of the present disclosure;

FIG. 9 illustrates a method of prescribing a drug or other therapy in touch-free medical treatment, according to an aspect of the present disclosure; and

FIG. 10 shows an exemplary general computer system that includes a set of instructions for touch-free medical treatment.

FIG. 11 illustrates a method for touch-free medical treatment with multi-stage artificial application, according to an aspect of the present disclosure.

FIG. 12 illustrates an overview process for touch-free medical treatment, according to an aspect of the present disclosure.

FIG. 13 illustrates a distribution of processes to be performed for touch-free medical treatment, according to an aspect of the present disclosure.

FIG. 14 illustrates a data flow for touch-free medical treatment, according to an aspect of the present disclosure.

FIG. 15 illustrates a process flow for touch-free medical treatment, according to an aspect of the present disclosure.

FIG. 16 illustrates a sequence of user interfaces for touch-free medical treatment, according to an aspect of the present disclosure.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below.

Methods described herein are illustrative examples, and as such are not intended to require or imply that any process of any embodiment be performed in the order presented. Words such as “thereafter,” “then,” “next,” etc. are not intended to limit the order of the processes, and these words are instead used to guide the reader through the description of the methods. Further, any reference to claim elements in the singular, for example, using the articles “a,” “an” or “the”, is not to be construed as limiting the element to the singular.

A touch-free medical treatment as described herein is a medical treatment for disease states that may be provided without a physical examination, and even without physical contact between a patient and a medical professional such as a doctor. A touchless medical treatment platform described herein enables patients to take laboratory tests; enter risk factors, health histories, and symptoms; undergo clinical assessments with healthcare providers; and obtain prescriptions for the given conditions, all from private settings such as in homes.

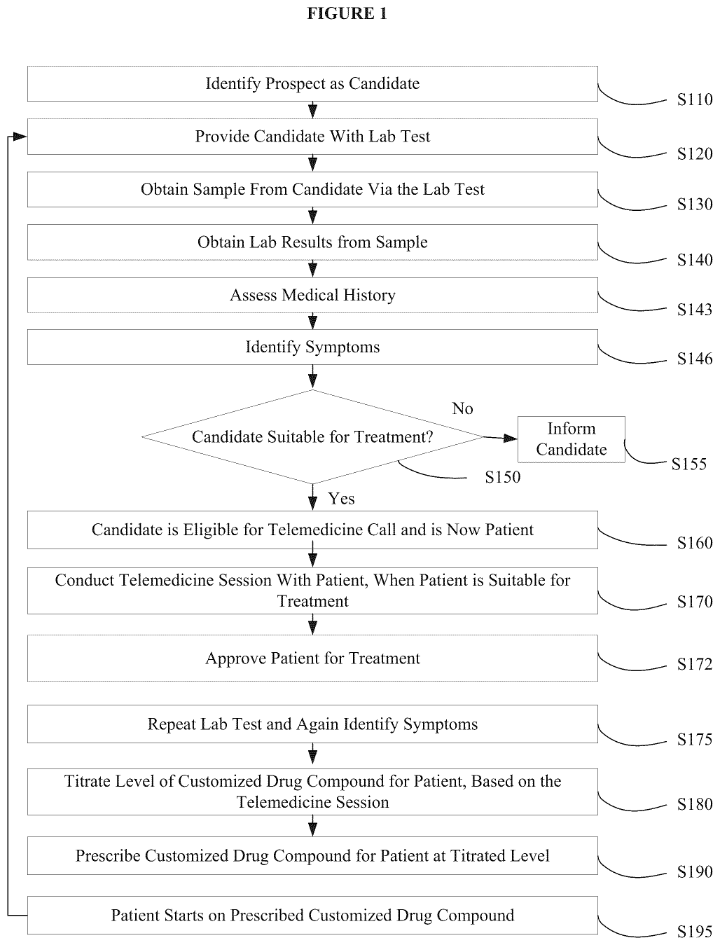

FIG. 1 illustrates a method for touch-free medical treatment, according to an aspect of the present disclosure.

In FIG. 1, the process starts at S110 by identifying a prospect as a candidate. The prospect may be a prospective patient who is identified based on internet searches by the prospect, phone contacts initiated by the prospect, emails initiated by the prospect, messages initiated by the prospect, and other forms of contact initiated by the prospect. In one example, prospects using search engines to search for specific terms such as “hormone replacement” or “hormone replacement therapy” may be automatically provided with results that include contact information (e.g., a phone number, email address, website) of a provider of the touch-free medical treatment described herein. The provider of the touch-free medical treatment described herein may also or alternatively be provided with information of prospects using the search engines to search for specific terms. The prospect becomes a candidate once identified by the provider of touch-free medical treatment at S110. Identifying a patient population may be important for developing and implementing targeted treatment interventions that meet the unique healthcare needs of different patient populations. Identifying the prospective patient can be derived from a patient candidate scoring system that looks through existing electronic medical records data base to identify candidates. The prospect may also be identified based on online data which may include, for example, demographic data, online behavioral data and online search data.

The process of FIG. 1 next proceeds with providing the candidate with a lab test at S120. The lab test may be provided to the candidate at an office, via a mail service or package delivery service, at a medical facility, or in any other feasible way. In an embodiment, the lab test may be an at-home test provided to the candidate to take at home (or a personal at-home lab analyzer using a microfluid chip or other analyzers) and then processed. The lab test may comprise a hormone lab panel.

After S120, the candidate may register a kit identification and provide additional health data. The additional health data may include health history and symptoms.

Next, the process of FIG. 1 includes obtaining a sample from the candidate via the lab test at S130. The candidate may collect a specimen and return the specimen to a lab or the sample is instantly or automatically processed or analyzed within the test kit, such as using a lab on a chip (also known as a microfluid chip) The sample from the candidate may be a biometric sample, and may be provided from the candidate via the lab test at an office, via return mail using a mail service or package delivery service, at a medical facility, or in any other way as applicable. In an embodiment, the lab test with the biometric sample is placed by the candidate in a return package and returned via the same mail service or package delivery service by which the lab test was initially provided at S120 (if applicable). A lab test may include a sealable vial, instructions, a padded package, a cotton swab or cotton ball, a syringe, or any other items conventionally used to capture and store or instantly process or analyze a biometric sample. A biometric sample provided from the candidate via the lab test may be sealed to prevent changes in oxygen concentration or other chemical characteristics due to exposure before lab results are obtained. As should be evident, the lab test may be provided to the patient and a biometric sample may be obtained from the patient without requiring a consultation with a doctor.

The process of FIG. 1 next proceeds by obtaining lab results from the sample at S140. The lab results may be obtained by processing the specimen from the candidate, such as to identify health markers such as estrogen levels, and determine whether the estrogen is too low by analyzing the sample returned via the lab test. Lab results may be performed by assaying the biometric sample. For example, portions of the biometric sample may be mixed with reagents, filtered, dried, weighed and analyzed. Alternatively or also, portions of the biometric sample may be subject to instrumental analysis, such as to characterize chemical reactions, measure properties of the biometric sample, and analyze the measured properties. For example, the sample may be a saliva sample, blood sample, urine sample, hair sample, or other form of biometric sample, and the lab results may be obtained by chemically analyzing the sample for characteristics indicative of a medical condition. Chemical analysis may determine levels of one or more hormones or biomarkers of the candidate. The determined levels of hormones or biomarkers may be compared to baseline expectations set for the candidate. Baseline expectations may vary for different candidates, such as based on genetic, demographic, and physical characteristics of different candidates.

An example of obtaining lab results is by saliva analysis, though lab results may also be based on analysis of blood, urine, stool, tissue, swabs and hair. Saliva testing may include laboratory analysis of saliva to identify markers of conditions such as endocrine conditions, immunologic conditions, inflammatory conditions, infectious conditions, and other types of conditions. Saliva can be used to assay steroid hormones, genetic material, proteins and antibodies. Saliva testing may be used to screen for or diagnose numerous medical conditions including Cushing's disease, cancer, parasites, hypogonadism, and allergies. Saliva testing is only one form of biometric testing that may be implemented using a lab test to obtain lab results at S140.

At S143, the process of FIG. 1 includes assessing the medical history of the candidate. The medical history may be assessed based on a medical history assessment form collected from the candidate. For example, a medical history assessment form may ask the candidate if the candidate has a history of cancer and/or other types of diseases. An assessment of any particular answer on a medical history assessment form may be determinative of whether the candidate is suitable for treatment, or may considered together with one or more other answers, such as by using a weighting system. A medical history assessment form may be provided via an interactive user interface over the internet, such as via a website provided at a uniform resource locator (URL) address. The candidate may be asked to establish a secure connection such as by creating an account and signing in, and then presented with the medical history assessment form to securely populate the medical history assessment form with medical information specific to the candidate. Alternatively, the medical history assessment form may be filled out over the phone by the candidate providing the medical information or at a facility (e.g., on paper submitted to a front desk).

At S146, the process of FIG. 1 includes collecting symptoms from the candidate. Symptoms may include, for example, hot flashes, fatigue, or other conditions and/or disease states that a candidate is experiencing. The symptoms may be provided also by the medical history assessment form, over the phone, or at a facility.

At S150, the system qualifies the candidate. The candidate may be qualified based on determinations of an absence of a history of cancer, confirmation of receipt of the lab test, and other information provided by or for the candidate. The determination at S150 may be made based on applying an algorithm that weights one or more characteristics of the sample and the candidate to obtain a score that is compared against a threshold. For example, the characteristics of the sample may include the presence or strength of one or more chemicals, compounds, bacteria species, virus species, antibodies, or other characteristics taken from the biological sample. The characteristics of the candidate may include health history such as a history of cancer, demographic characteristics age, race, gender, weight, height, genetic characteristics, family history, answers provided from the candidate via the medical history assessment form or other forms of information that may be indicative of a medical condition. The characteristics of the sample and of the candidate may also indicate a relatively low risk, a relatively high risk, a relatively mild infection, a relatively serious infection, a relatively mild illness, a relatively serious illness, or other relative factors that indicate the presence and/or seriousness of a medical condition.

The determination at S150 may be based on applying a trained artificial intelligence model to determine the most appropriate treatment(s) (e.g., FDA approved drug, compounded drug formulation, OTC medication, dietary supplement, lifestyle treatment, etc.) across disease states treated including but not limited to hormone-related issues (e.g., menopause, perimenopause, andropause, PCOS, infertility, hypothyroidism, adrenal fatigue, etc.), dermatological conditions (e.g., melasma, acne, rosacea, alopecia, etc.), sexual dysfunction, mental health disorders (e.g., depression, anxiety, etc.), metabolic disorders (e.g., weight gain, etc.), cardiovascular conditions, musculoskeletal issues, neurological conditions, infectious diseases, and autoimmune conditions. The trained artificial intelligence model may be applied at S150 to identify treatment for disease states caused by menopause and perimenopause based on applying the trained artificial intelligence model to patient data. The trained artificial intelligence model that may be applied at S150 may be a first trained artificial intelligence model. Characteristics of the patient such as demographic information of the patient, a medical history of the patient, and other types of data may be input to the first trained artificial intelligence model. The first trained artificial intelligence model may be trained using a large number of data sets of historical patients who have been successfully and/or unsuccessfully treated to identify weights for the various types of data that can be collected as inputs for the first trained artificial intelligence model. The first trained artificial intelligence model may also be retrained, such as periodically or intermittently every 3 or 6 months, to update and improve the predictions output from the first trained artificial intelligence model.

Artificial intelligence models include machine learning as a category. Machine learning is trained based on data. Machine learning (ML) solutions often involve the detection of a particular condition (positive class) among a number of instances of alternative conditions which include an opposite condition (negative class). As noted, the first trained artificial intelligence model may be trained using a large number of data sets of historical patients who have been successfully and/or unsuccessfully treated, to identify weights for the various types of data that can be collected as inputs for the first trained artificial intelligence model. Examples of the inputs to the first trained artificial intelligence model may include the above-noted characteristics of the sample and/or the above-noted characteristics of the candidate, and the first trained artificial intelligence model may have been trained using datasets that include corresponding characteristics for previous candidates. A machine learning component of touch-free medical treatment as described herein allows for improved healthcare treatment by assisting in diagnostics, treatment determinations, drug selection optimization, and generally reduces medical errors and improves patient outcomes through predictive analytics. By analyzing health data, machine learning algorithms can identify patterns and make predictions based on symptoms, laboratory results and health history. Data processed by a machine learning component may include lab test results, symptoms, health history, and risk factors, and the machine learning component may be configured to formulate decisions for clinical treatment flows and personalized medicine formulations to predict improved patient outcomes.

If the candidate is not suitable for treatment (S150=No), at S155 the candidate is informed and the process ends. For example, the candidate may be called, emailed or messaged to inform the candidate that the candidate is not suitable for treatment. The candidate may be deemed unsuitable based on the first trained artificial intelligence model predicting that the candidate has a low risk or low deficiency of one or more hormones or biomarkers.

If the candidate is suitable for treatment (S150=Yes), the candidate becomes a patient at S160. The candidate becoming a patient at S160 means that the candidate will be treated by a medical professional and/or with medicine and/or with a medical procedure. The candidate may be deemed suitable based on the first trained artificial intelligence model predicting that the candidate has a high risk or high deficiency or one or more hormones or biomarkers.

At S170 a telemedicine session is conducted with the patient. The telemedicine session may be arranged so that the patient uses a telephone, computer and/or television to electronically interface with a professional in order to discuss the lab results and any other relevant information that may be pertinent in treating the patient for a medical condition. The professional may be a doctor, a nurse practitioner, a pharmacist, a dentist, or another appropriate professional appropriate for a discussion with the patient. The telemedicine session may be performed using the internet. The telemedicine session may use video cameras, audio microphones, screen-sharing programs, document-sharing programs, and other electronic mechanisms for two (or more) individuals to communicate over electronic networks.

The telemedicine session at S170 may be synchronous or asynchronous, such as via video, a call, email and/or text. The telemedicine session at S170 may be driven by an artificial intelligence model which allows providers to streamline a diagnosis process, inform clinical decision-making and help drive improvements to patient care. Medical practitioners can use the artificial intelligence model utilizing empirical evidence from an entire patient population to inform their treatment decisions, rather than relying on anecdotal evidence.

At S175, the process of FIG. 1 includes repeating the lab test and checking with the patient for updated symptoms. The lab test repeated at S175 may be simply to confirm the results of the earlier lab test. The check for updated symptoms at S175 may be performed to see if any symptoms have begun or ended since the collection of symptoms at S146. The lab test repeated at S175 may be repeated in the same manner as the lab test performed to obtain lab results from the biometric sample at S140. However, at S175 the candidate may provide the biometric sample remotely such as via return mail, or at a facility such as a medical facility. In some embodiments, S175 is omitted.

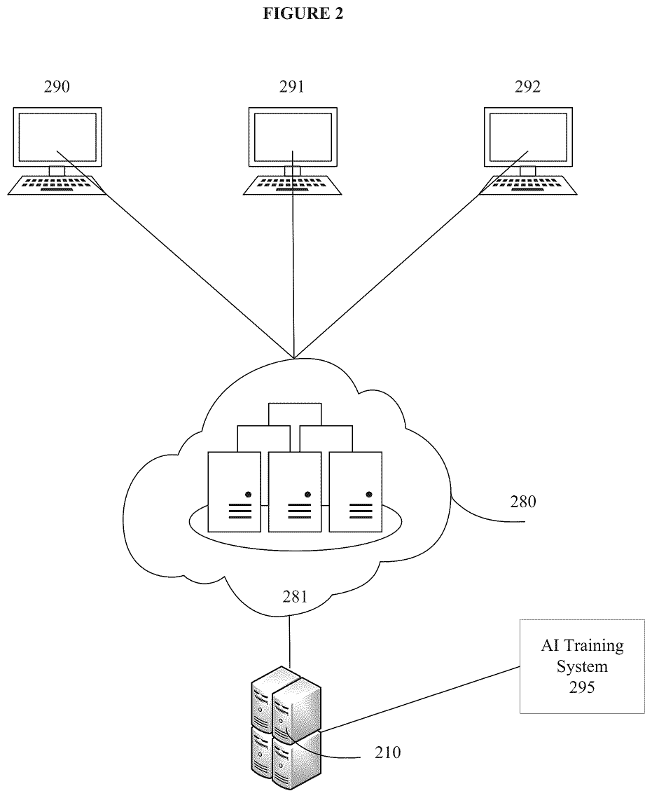

After the telemedicine session at S170, an artificial intelligence model may be applied to recommend a treatment. Treatment may be recommended based on indicated symptom severity, lab levels, weight, monthly cycle, and the treatment may comprise a customized prescription. The server 210 or a system which includes the server 210 in FIG. 2 may apply an artificial intelligence model to the information provided by or for the candidate/patient, and may recommend the treatment.

At S180, the process of FIG. 1 includes titrating a level of a customized drug or other therapy for the patient, based on the telemedicine session. The titration process may involve adding or removing a drug to the patient's treatment plan through single and combination therapy. The titrating at S180 may be performed based on applying a trained artificial intelligence model to determine the dosage and formulation of the customized drug such as a compound. The trained artificial intelligence model that may be applied at S180 may be a second trained artificial intelligence model. The second trained artificial intelligence model may be trained based on results of treating a plurality of additional patients before the patient. The second trained artificial intelligence model may be trained based on results of customizing the customized drug for a plurality of additional patients treated before the patient. For example, the second trained artificial intelligence model may receive as inputs levels of hormones or other biomarkers of the patient, so that the levels can be adjusted by the customized drug or other treatment in a manner that remedies deficiencies relative to a baseline.

The level of the customized drug such as a compound may be titrated specifically to remedy a medical condition experienced by the patient, and may be titrated based on the telemedicine session, the lab results, and may be titrated based on a trained artificial intelligence model applied to information of condition experiences by a set of other patients.

The customized drug may be bioidentical hormone replacement therapy (BHRT). Bioidentical hormone replacement therapy may also be known as bioidentical hormone therapy or natural hormone therapy, and involves applying hormones that are identical on a molecular level with hormones produced by the body. The customized drug or other therapy may be applied specifically to return balance to the patient's hormone levels.

At S190, the process of FIG. 1 includes prescribing the customized drug for the patient at the titrated level. The customized drug may be prescribed by the professional who conducts the telemedicine session at S170. Alternatively, in jurisdictions where prescriptions can be made automatically, the customized drug may be prescribed automatically. The customized drug may be prescribed without face-to-face contact with a medical professional to obtain treatment via the customized drug.

In an embodiment, the prescription at S190 may be performed before the titrating at S180, such as in places where titrating cannot be initiated before a specific prescription.

After the patient purchases the prescription from S190, the patient may start the prescription. Afterwards, the patient may repeat the data collection process from S120 to determine the effectiveness of the treatment prescribed at S190. For example, the repeated process may be used to identify symptom changes and lab level changes.

As further explanation of the variables that may be considered in the process of FIG. 1, hormone deficiency is an example medical condition that may be addressed. Hormone deficiency in some women, particularly women at or near menopause, can be addressed by hormone replacement therapy (HRT). Hormone replacement therapy can help balance estrogen, progesterone, testosterone, levothyroxine, liothyronine, dehydroepiandrosterone (DHEA), and cortisol levels, for example. Of course, hormone therapies are not limited to women, to menopause, or to replacement. For women at or near menopause, hormone replacement therapy may alleviate symptoms such as sweating and hot flashes, and may also reduce (or increase) risks of some other conditions such as osteoporosis. Additionally, the hormone replacement therapy may include one compound such as estrogen (e.g., estradiol and estriol), or may include multiple compounds such as progesterone and estrogen.

As set forth above with respect to the method of FIG. 1, a platform may be configured to automatically and instantaneously dropship lab tests as in S120. Users may be prompted to take the lab tests and return samples from the lab tests for S130. Data or information may be requested from the user for S143. The data may be stored and presented in a simple or graphical manner. The data may be evaluated at S143. The patients may be provided with patient education, and may arrange for clinical appointments at specific moments in the treatment cycle. The use of telemedicine sessions at S170 may reduce or eliminate the need for manual intervention and may drive the continued user experience.

Using a method such as the method of FIG. 1, test results and symptom improvement may be tracked over time within each patient's profile. The data collected over time across all patient profiles may be analyzed collectively for patient efficacy and safety, providing a modality to establish a standard of care model for personalized medicine treatments. FIG. 2 illustrates a networked arrangement for touch-free medical treatment, according to an aspect of the present disclosure.

In FIG. 2, the network includes a first user computer 290, a second user computer 291, a third user computer 292, a communications network 280 that includes a data center 281 therein, a server 210, and an AI training system 295. Each of the first user computer 290, the second user computer 291 and the third user computer 292 may belong to and may be used by completely separate individuals. The individuals using the first user computer 290, the second user computer 291 and the third user computer 292 may be using a search engine via a browser in order to find information on a topic related to a medical condition.

Any or all of the first user computer 290, the second user computer 291 and/or the third user computer 292 may be a smart phone, a laptop computer, a stationary computer, or any other type of computing and communications device used by users.

The data center 281 is representative of networked computers used to implement cloud services for end users. Networked computers may be used to provide one or more trained artificial intelligence model(s) as described herein, such as to optimize determinations as to whether a prospect would be an appropriate candidate for lab testing, whether a candidate would be an appropriate patient for a medical treatment, and which compounds and which levels of the compound(s) would be optimal for treating a patient.

The server 210 is representative of a computer or computer system (i.e., with multiple networked computers) which manages one or more aspects of touch-free medical treatment as described herein. A computer or computer system which includes the server 210 may comprise a component of a touchless medical treatment platform described herein, and may provide services such as to analyze lab test samples, risk factors, health histories and symptoms and other types of information from prospective patients. The touchless medical treatment platform may also interact with medical personnel such as to collect questions to present to prospective patients and to receive prescriptions or other therapies for given conditions. The touchless medical treatment platform may also analyze data for one prospective patient or for some or all prospective patients as a group, such as to optimize outcomes based on medical data and demographic data from different prospective patients. For example, the server 210 may coordinate with providers of search engines in order to identify prospects as candidates, analyze lab results from samples to determine whether candidates are suitable for treatment, initiate scheduling of telemedicine sessions to determine whether candidates are suitable for treatment. Additionally, a server 210 may also or alternatively be used to provide trained artificial intelligence models as described herein, such as to optimize determinations as to whether a prospect would be an appropriate candidate for lab testing, whether a candidate would be an appropriate patient for a medical treatment, and which treatments would be optimal for treating a patient.

In an embodiment, the server 210 may manage a relationship with cloud services that include the data center 281 in order to provide the trained artificial intelligence models as described herein. The server 210 may provide divided services on behalf of the provider of touch-free medical treatment. The divided services may provide one portal for candidates and/or patients, and another portal on behalf of healthcare providers. The portals may be provided via a website.

The AI training system 295 is representative of a source of a first trained artificial intelligence model and a second trained artificial intelligence model, and may be entirely separate from the server 210 which applies these models. To train a first trained artificial intelligence model described herein, machine learning may be applied by the AI training system 295 to data from results of treating a plurality of patients, including patients who were treated successfully and/or patients who were not treated successfully. As a result, when the first trained artificial intelligence model is applied at S150 in FIG. 1, the first trained artificial intelligence model may have been trained based on results of treating a plurality of additional patients treated before the current patient.

To train a second trained artificial intelligence model described herein, machine learning may be applied by the AI training system 295 to data from results of treating a plurality of patients, including patients with varying characteristics who were treated with a variety of titrated levels of a customized drug or other therapy. As a result, when the second trained artificial intelligence model is applied at S180 in FIG. 1, the second trained artificial intelligence model may have been trained based on results of customizing the customized drug or other therapy for a plurality of additional patients treated before the current patient.

Training of models by the AI training system 295 may involve aggregating correlations across multiple inputs to identify candidates who are suitable for treatment (i.e., the first trained artificial intelligence model) and to identify an appropriate titration level of a customized drug or other treatment (i.e., the second trained artificial intelligence model). Inputs to both the first trained artificial intelligence model and the second trained artificial intelligence model may include demographic characteristics and the lab results from the candidate.

In some embodiments, patients may be treated over a lengthy period of time before artificial intelligence models are finalized and used to generate or used to assist in generation of treatment recommendations. For example, data may be collected and anonymized for thousands of patients treated using the touch-free medical treatment, and then a variety of data from treating a variety of patients is used to train an artificial intelligence model. Patient data outcomes may be used in the process of training a machine learning model. For example, inputs used in training may comprise symptoms and demographic characteristics and lab levels along with successful and unsuccessful treatments.

Using the AT training system 295, the system 200 leverages machine learning to process and evaluate data such as test results, symptoms, health history, and risk factors. The system 200 is enabled to formulate improved decisions for clinical treatment flows and personalized medicine formulations to predict high quality patient outcomes.

Additionally, the system 200 uses machine learning to identify the most effective and safe compounded drugs or other treatments across any and all disease states treated, including but not limited to hormone-related issues (e.g., menopause, perimenopause, andropause, PCOS, infertility, hypothyroidism), dermatological conditions (e.g., melasma, acne, rosacea, alopecia), sexual dysfunction, mental health disorders (e.g., depression, anxiety), metabolic disorders (e.g., weight gain, insulin resistance), autoimmune conditions, etc.

The AI training system 295 may be trained using training sets such as data of lab ranges and dosage ranges. Algorithms may be specified for use in the AI training system. Examples of potentially appropriate algorithms include regression algorithms, classification algorithms, decision trees and neural networks. Benchmarks may be defined to evaluate each machine learning model. One or more models may be selected in a refinement process, and then the selected machine learning model may be deployed as a trained artificial intelligence model from the AI training system 295.

The system 200 may be used to collect and aggregate clinical evidence from patients so as to prove the effectiveness and safety profile of compounded drugs in a manner similar to what would be appropriate for a drug approved by the United States Food and Drug Administration.

Using a networked arrangement as in FIG. 2, patients can access their health information, communicate with their healthcare providers, and manage their health from the comfort of their own homes. Patients may be enabled to track and monitor their health data in real-time. Healthcare providers are enabled to access more comprehensive and up-to-date information about their patients. Additionally, a networked arrangement as in FIG. 2 is only one example of the Artificial Intelligence (AI) training system amplifying a touch-free medical treatment which may be provided as a transformative service for patients.

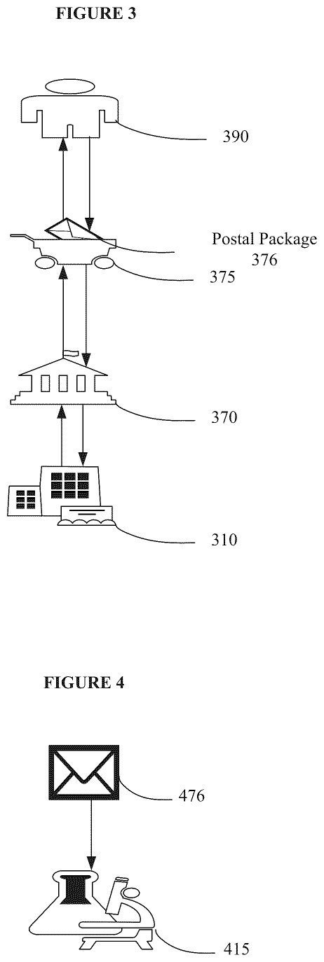

FIG. 3 illustrates a flow of movement for a lab test in touch-free medical treatment, according to an aspect of the present disclosure.

In FIG. 3 the movement starts at a distribution facility 310 and flows to a postal facility 370, to a postal delivery vehicle 375 that includes a postal package 376, to an end user 390, and returns from the end user 390 (if applicable), to the postal delivery vehicle 375, to the postal facility 370, to the distribution facility 310. More particularly, a distribution facility 310 is used to source a postal package 376 with a lab test that is sent to the postal facility 370. The postal package 376 moves through the mail system as represented by the postal delivery vehicle 375. The postal package 376 is provided to the end user 390. The end user provides a biometric sample to the lab test in the postal package 376, and returns the postal package 376 by return mail through the mail system as represented by the postal delivery vehicle 375 (if applicable). The postal package 376 returns to the distribution facility 310 via the postal facility 370.

In another embodiment, a lab test may be provided to the end user 390 at a medical facility such as a clinic. In some embodiments, the biometric sample may be realized in real-time or near real-time so that the determination of whether the end user 390 as a candidate is suitable for treatment at S150 may be made in real-time or near real-time. For example, the end user 390 may visit a clinic at an appointed time or as a walk-in, and be provided with the lab test to check whether the end user 390 as a candidate is suitable for treatment.

FIG. 4 illustrates a method of performing a laboratory analysis from a lab test in touch-free medical treatment, according to an aspect of the present disclosure.

In FIG. 4, the method includes providing the lab test from a postal package 476 to lab equipment 415. The lab equipment 415 analyzes the lab test. In some embodiments, the lab test kit contains reagents or other elements to auto-process the sample on demand. More particularly, the lab equipment 415 is representative of equipment used to analyze a biometric sample provided from an end user 390 as a candidate. The biometric sample may be any of a blood sample, a urine sample, a hair sample, a saliva sample, or any other type of biometric sample that may be analyzed for information indicative of a medical condition for which a prospect seeks treatment.

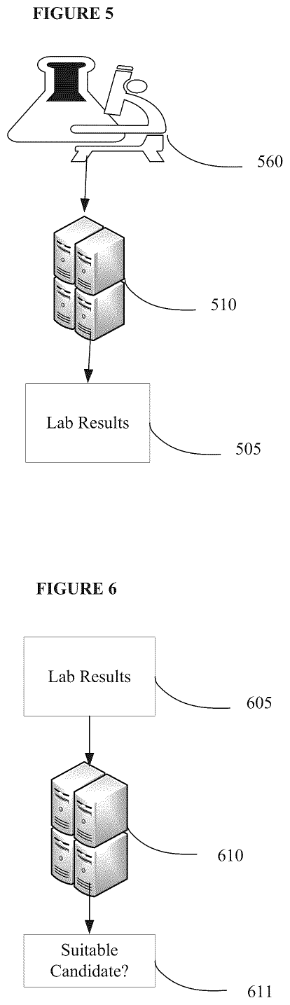

FIG. 5 illustrates a method of obtaining lab results from a laboratory analysis in touch-free medical treatment, according to an aspect of the present disclosure.

In FIG. 5, the method includes outputting results of the lab analysis from the lab equipment 560 to a server 510 and generating lab results 505. The server 510 may analyze the raw analytical results from lab equipment 560 to generate lab results 505. The lab results 505 may reflect an imbalance (excess or deficiency) of a hormone, a vitamin, a mineral, cell type, antibody, or another type of biomarker typically found in a human body. The results may also reflect the presence or absence of a bacteria or virus.

A computer or computer system which includes the server 510 may comprise a component of a touchless medical treatment platform described herein, and may provide services such as to analyze lab test samples, risk factors, health histories and symptoms and other types of information from prospective patients. The touchless medical treatment platform may also interact with medical personnel such as to collect questions to present to prospective patients and to receive prescriptions or other treatments for given conditions. The touchless medical treatment platform may also analyze data for one prospective patient or for some or all prospective patients as a group, such as to optimize outcomes based on medical data and demographic data from different prospective patients.

FIG. 6 illustrates a method of identifying a suitable candidate from lab results in touch-free medical treatment, according to an aspect of the present disclosure.

In FIG. 6, the method includes inputting the lab results 605 to a server 610 and identifying whether the patient is a suitable candidate. The lab results 605 may provide absolute or relative levels of biomarkers, and/or presence or absence of a bacteria or virus. The server 610 may apply a trained artificial intelligence model to the lab results 605 to generate a recommendation whether the candidate is suitable for treatment as a suitable candidate determination 611. The recommendation may be based on previous recommendations and treatment results from multiple previous candidates subjected to lab testing of biometric samples for the same medical condition.

A computer or computer system which includes the server 610 may comprise a component of a touchless medical treatment platform described herein, and may provide services such as to analyze lab test samples, risk factors, health histories and symptoms and other types of information from prospective patients. The touchless medical treatment platform may also interact with medical personnel such as to collect questions to present to prospective patients and to receive prescriptions for given conditions. The touchless medical treatment platform may also analyze data for one prospective patient or for some or all prospective patients as a group, such as to optimize outcomes based on medical data and demographic data from different prospective patients.

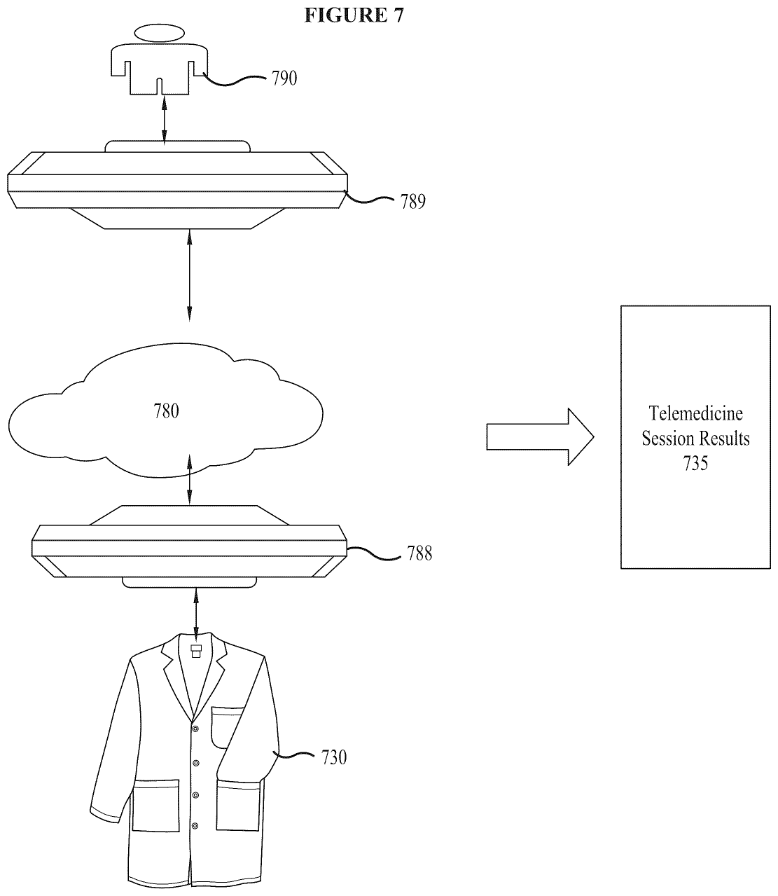

FIG. 7 illustrates a method of conducting a telemedicine session with a touch-free medical treatment, according to an aspect of the present disclosure.

In FIG. 7, the method includes conducting a telemedicine session between a patient 790 and a medical professional 730 over an electronic communication network 780 to produce telemedicine session results.

The telemedicine session in FIG. 7 may be conducted so that a medical professional can be provided with the lab results 505 and a record of medical history from the patient, and interact with the patient to dynamically solicit additional information from the patient that would be relevant to generating or confirming a treatment plan for the patient. The medical professional may be provided information relating to the patient from the server 210, including the initial information relating to the inquiry when the patient was a prospect, the analysis of the lab results provided when the candidate submitted the biometric sample via the lab test, and any additional information provided by the patient, an insurance company for the patient, other medical providers for the patient, or any other information sources that will help the medical professional generate or confirm a plan for medical treatment.

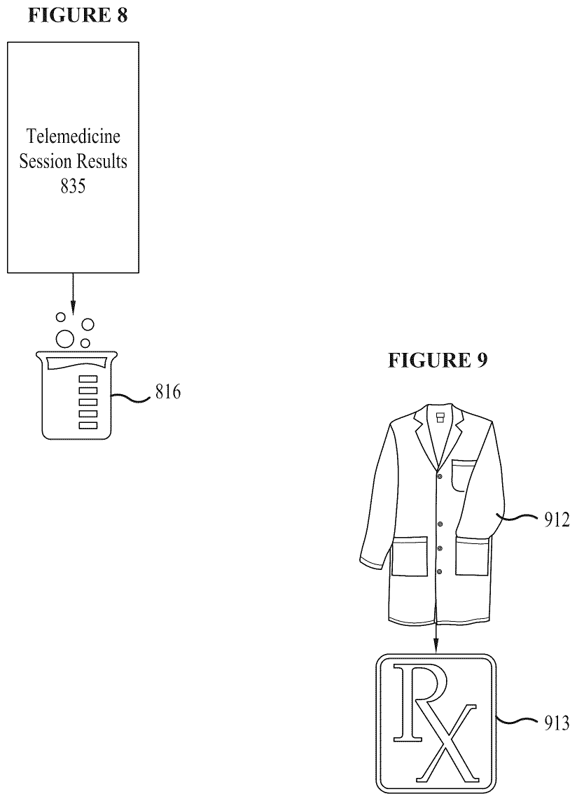

FIG. 8 illustrates a method of titrating a level of a customized drug or other treatment in touch-free medical treatment, according to an aspect of the present disclosure.

The titration process may involve adding or removing a drug to the patients treatment plan through single and combination therapy. In FIG. 8, the method includes using the telemedicine session results as a basis for titrating a level of a customized drug or other treatment using titration equipment 816. In an embodiment, the titration in FIG. 8 may involve selecting absolute or relative levels of one or more compounds that will be provided to the patient on a treatment schedule. The absolute or relative levels may be determined using a trained artificial intelligence model that is trained based on previous titrations and patient outcomes for multiple previous patients. The trained artificial intelligence model may be implemented by a server such as the server 210, the server 510, or the server 610, or in a cloud that includes the data center 281.

FIG. 9 illustrates a method of prescribing a customized drug or other therapy in touch-free medical treatment, according to an aspect of the present disclosure.

In FIG. 9, the method includes a medical professional 912 prescribing the customized drug or other therapy to the patient for treatment. In an embodiment, the medical professional 912 may endorse levels of one or more compounds generated by a server such as the server 210, the server 510, or the server 610, or in the cloud that includes the data center 281 as the prescription. In another embodiment, the medical professional 912 may be replaced by a “smart” system when an automated system is allowed by law to initiate prescriptions or other therapies based on predetermined instructions and a trained artificial intelligence model.

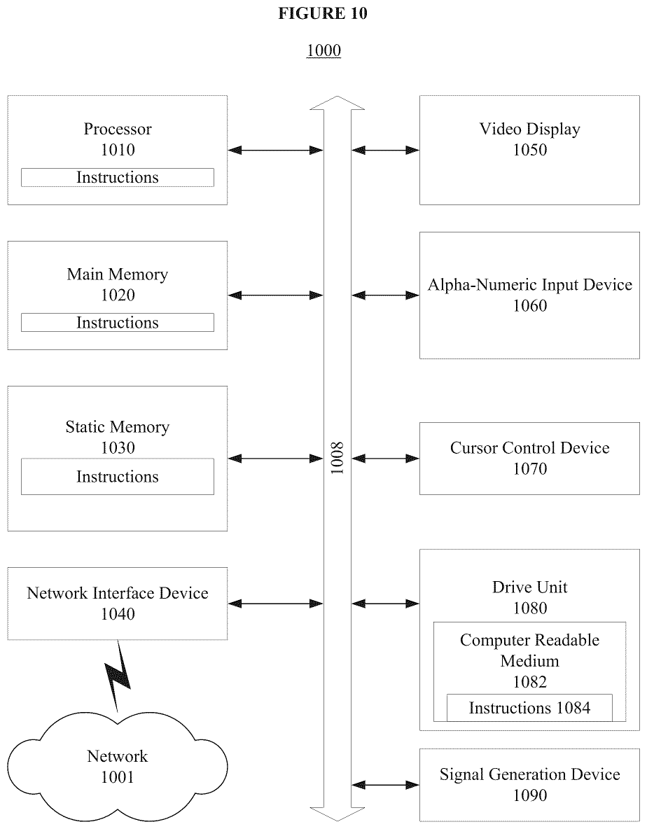

FIG. 10 illustrates an exemplary general computer system that includes a set of instructions for touch-free medical treatment.

FIG. 10 illustrates a computer system, on which a method for touch-free medical treatment is implemented, in accordance with another representative embodiment.

The computer system 1000 of FIG. 10 shows a complete set of components for a communications device or a computer device. The computer system 1000 may include some or all elements of one or more component apparatuses in a system for touch-free medical treatment herein, although any such apparatus may not necessarily include one or more of the elements described for the computer system 1000 and may include other elements not described.

Referring to FIG. 10, the computer system 1000 includes a set of software instructions that can be executed to cause the computer system 1000 to perform any of the methods or computer-based functions disclosed herein. The computer system 1000 may operate as a standalone device or may be connected, for example, using a network 1001, to other computer systems or peripheral devices. In embodiments, a computer system 1000 performs logical processing based on digital signals received via an analog-to-digital converter.

In a networked deployment, the computer system 1000 operates in the capacity of a server or as a client user computer in a server-client user network environment, or as a peer computer system in a peer-to-peer (or distributed) network environment. The computer system 1000 can also be implemented as or incorporated into various devices, such as the server 210 in FIG. 2, the server 510 in FIG. 5 or the server 610 in FIG. 6, a stationary computer, a mobile computer, a personal computer (PC), a laptop computer, a tablet computer, or any other machine capable of executing a set of software instructions (sequential or otherwise) that specify actions to be taken by that machine. The computer system 1000 can be incorporated as or in a device that in turn is in an integrated system that includes additional devices. In an embodiment, the computer system 1000 can be implemented using electronic devices that provide voice, video or data communication. Further, while the computer system 1000 is illustrated in the singular, the term “system” shall also be taken to include any collection of systems or sub-systems that individually or jointly execute a set, or multiple sets, of software instructions to perform one or more computer functions.

As illustrated in FIG. 10, the computer system 1000 includes a processor 1010. The processor 1010 may execute instructions to implement some or all aspects of methods and processes described herein. The processor 1010 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1010 is an article of manufacture and/or a machine component. The processor 1010 is configured to execute software instructions to perform functions as described in the various embodiments herein. The processor 1010 may be a general-purpose processor or may be part of an application specific integrated circuit (ASIC). The processor 1010 may also be a microprocessor, a microcomputer, a processor chip, a controller, a microcontroller, a digital signal processor (DSP), a state machine, or a programmable logic device. The processor 1010 may also be a logical circuit, including a programmable gate array (PGA), such as a field programmable gate array (FPGA), or another type of circuit that includes discrete gate and/or transistor logic. The processor 1010 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1000 further includes a main memory 1020 and a static memory 1030, where memories in the computer system 1000 communicate with each other and the processor 1010 via a bus 1008. The main memory 1020 and the static memory 103 are each examples of a tangible non-transitory computer readable storage medium, and may store instructions used to implement some or all aspects of methods described herein. Either or both of the main memory 1020 and the static memory 1030 may store instructions used to implement some or all aspects of methods and processes described herein. Each memory described herein is a tangible, non-transitory, computer readable storage medium for storing data and executable software instructions such as computer programs and sub-programs. The memories are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1020 and the static memory 1030 are articles of manufacture and/or machine components. The main memory 1020 and the static memory 1030 are computer-readable mediums from which data and executable software instructions can be read by a computer (e.g., the processor 1010). The executable instructions of a computer program may cause a computer apparatus to implement one or more process as described herein. Each of the main memory 1020 and the static memory 1030 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted. [097]“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1000 further includes a video display unit 1050, such as a liquid crystal display (LCD), an organic light emitting diode (OLED), a flat panel display, a solid-state display, or a cathode ray tube (CRT), for example. Additionally, the computer system 1000 includes an input device 1060, such as a keyboard/virtual keyboard or touch-sensitive input screen or speech input with speech recognition, and a cursor control device 1070, such as a mouse or touch-sensitive input screen or pad. The computer system 1000 also optionally includes a disk drive unit 1080, a signal generation device 1090, such as a speaker or remote control, and/or a network interface device 1040.

In an embodiment, as depicted in FIG. 10, the disk drive unit 1080 includes a computer-readable medium 1082 in which one or more sets of software instructions 1084 (software) are embedded. The sets of software instructions 1084 are read from the computer-readable medium 1082 to be executed by the processor 1010. Further, the software instructions 1084, when executed by the processor 1010, perform one or more steps of the methods and processes as described herein. In an embodiment, the software instructions 1084 reside all or in part within the main memory 1020, the static memory 1030 and/or the processor 1010 during execution by the computer system 1000. Further, the computer-readable medium 1082 may include software instructions 1084 or receive and execute software instructions 1084 responsive to a propagated signal, so that a device connected to a network 1001 communicates voice, video or data over the network 1001. The software instructions 1084 may be transmitted or received over the network 1001 via the network interface device 1040.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

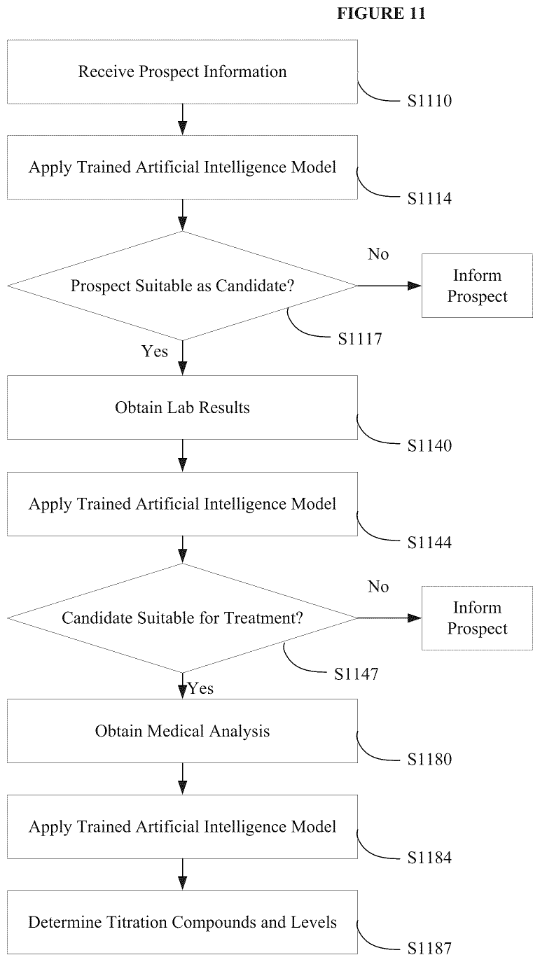

FIG. 11 illustrates a method for touch-free medical treatment with multi-stage artificial application, according to an aspect of the present disclosure.

In FIG. 11, prospect information is received at S1110. The prospect information may be information that is collected based on an internet query such as via a search engine or a visit to a website. The information may be a query for information relating to a medical condition, and may also include information collected from the prospective patient or their representative who is making the query. The information may include basis demographic and symptom information.

At S1114, a trained artificial intelligence model is applied to the prospect information to determine whether the prospect is suitable as a candidate. The trained artificial intelligence model may be applied based on previous sets of prospect information and resulting lab results from the previous prospective patients. For example, prospective patients describing symptom information inconsistent with the medical condition they are inquiring about may be identified as having a very low likelihood of having lab results reflective of the medical condition. In another example, prospective patients describing demographic information inconsistent with the medical condition they are inquiring about may be identified having a very low likelihood of having lab results reflective of the medical condition.

At S117, a determination is made whether the prospect is suitable as a candidate. If the prospect is not suitable (S1117=No), the prospect is informed. If the prospect is suitable (S1117=Yes), the process continues.

At S1140, lab results are obtained. The lab results may be obtained based on a lab test that includes a biometric sample from the candidate.

At S1114, the trained artificial intelligence model is applied to the lab results to determine whether the candidate is suitable for medical treatment for a diagnosable medical condition. The trained artificial intelligence model may be applied based on previous sets of lab results and resulting medical treatments for candidates treated based on the lab results. For example, candidates with chemical, bacterial or viral profiles inconsistent with successful treatments for the medical conditions for which the candidates seek treatment may be identified as having a very low likelihood of successfully remedying the medical conditions with the treatment. In another example, candidates with health information (e.g., weights, other medical conditions) inconsistent with successful treatments for the medical conditions for which the candidates seek treatment may be identified as having a very low likelihood of successfully remedying the medical conditions with the treatment.

At S1147, a determination is made whether the candidate is suitable for treatment. If the candidate is not suitable (S1147=No), the candidate is informed. If the prospect is suitable (S1147=Yes), the process continues.

At S1180, a medical analysis is obtained from the patient. For example, the medical analysis may be obtained by a medical professional such as a doctor, or by a computer when a computer is authorized to provide such a medical analysis. The medical analysis may include information such as demographic information, candidate medical history obtained from the patient or from previous medical reports and medical analyses, information resulting from the initial query by the patient when the patient was a prospect, and the lab results.

At S1184, a trained artificial intelligence model is applied to the medical analysis to determine the optical treatment for the medical condition to be treated. For example, optimal treatments may include a selection of one or more prescription or non-prescription drug, dosage of each drug, and frequency of treatment (e.g., daily, every 4 hours, every 12 hours) using the one or more prescription or non-prescription drug. The treatments may be optimized based on previous treatments and resulting medical outcomes for patients previously treated for the medical condition. The optimal treatment may include one or more prescription or non-prescription drug provided at dosages and at a frequency most likely to result in a positive outcome based on the analysis by the trained artificial intelligence model that takes into account the previous treatments and resulting medical outcomes for previous patients.

Accordingly, multiple trained artificial intelligence models may be incorporated into touch-free medical treatment in order to confirm whether a prospect is a suitable candidate, to confirm whether a candidate is a suitable patient, and to identify an optimal treatment for a candidate.

As set forth above, a prospective patient may be provided with medical treatment without a physical examination and even without physically contacting a doctor or other medical professional. The approval of a prospective patient for testing may be based on a trained artificial intelligence model. The analysis of lab testing results may be performed using a trained artificial intelligence model. Additionally, the determination of absolute or relative regimens of prescription or non-prescription drug to use in treating a patient may be performed using a trained artificial intelligence model. The medical treatments may be customized for patients using a trained artificial intelligence model that takes into consideration a variety of factors and treatments and outcomes from patients previously treated for the same medical condition.

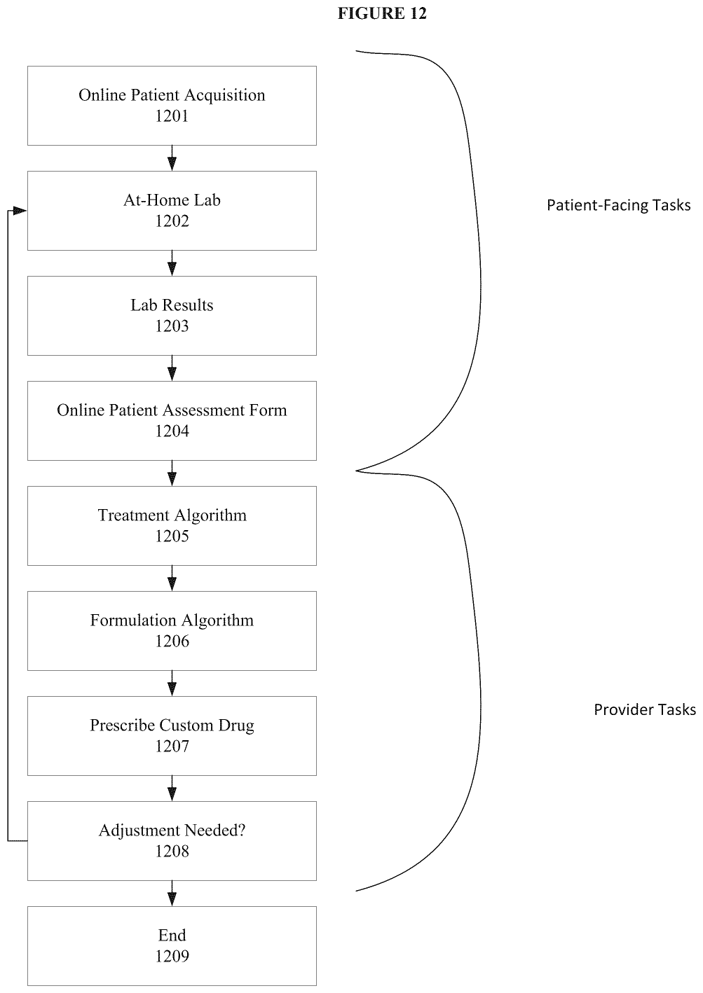

FIG. 12 illustrates an overview process for touch-free medical treatment, according to an aspect of the present disclosure.

In FIG. 12, processes are divided between patient-facing tasks and provider tasks. The patient-facing tasks include online patient acquisition 1201, an at-home lab test 1202, lab results 1203, and an online patient assessment form 1204. The patient-facing task group in FIG. 12 includes tasks related to adherence to a medical protocol for a given therapy.

The provider tasks include a treatment algorithm 1205, a formulation algorithm 1206, a prescribe custom drug task 1207, an adjustment needed determination task 1208, and an end task 1209. If an adjustment is needed according to the adjustment needed determination task 1208, the process returns to the at-home lab test 1202.

In the online patient acquisition 1201, patients may be prescreened via online targeting. At 1202, 1203 and 1204, diagnostic and screening methods may be applied. At 1205, artificial intelligence may be applied to identify and categorize prime candidates suitable for treatment. At 1206, artificial intelligence may be applied to formulate the appropriate compound using a treatment algorithm along with a broader integrated clinical and scientific guidelines, to allow for more reliable dosage forms and safer, effective medicines usage in target cohorts. For example, a list of drug formulations or treatment regimens with the greatest potential for treatment in a target cohort may be identified. The artificial intelligence at 1206 may determine the correct measurement and mix for dispensing to individual patients in the target cohort. The prescription at 1207 may include an online fulfillment of the customized drug or other therapy.

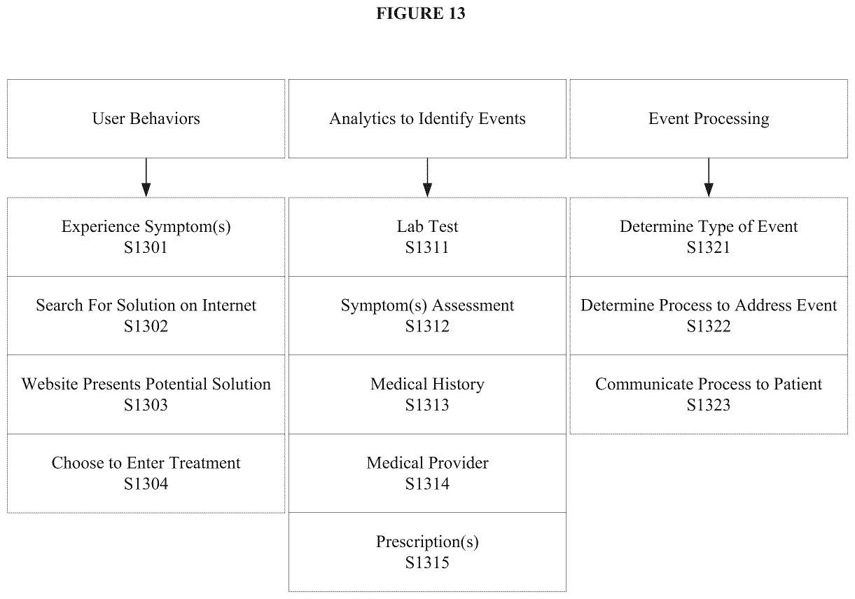

FIG. 13 illustrates a distribution of processes to be performed for touch-free medical treatment, according to an aspect of the present disclosure.

In FIG. 13, processes to be performed for touch-free medical treatment are distributed into three groups. The first group includes user behaviors, the second group includes analytics to identify events, and the third group include event processing.

The user behaviors group includes a user experiencing one or more symptom(s) at S1301. At S1302, the user searches for a solution on the internet. At 51303, the user is presented with a potential solution at a website. At S1304, the patient chooses to enter treatment.

The analytics group includes a lab test at S1311, an assessment of one or more symptom(s) at S1312, an analysis of medical history at S1313, an analysis of medical provider(s) at S1314, and an analysis of one or more prescription(s) at S1315.

The event processing group include determining a type of event at 51321, determining a process to address the event at S1322, and communicating the process to the patient at S1323.

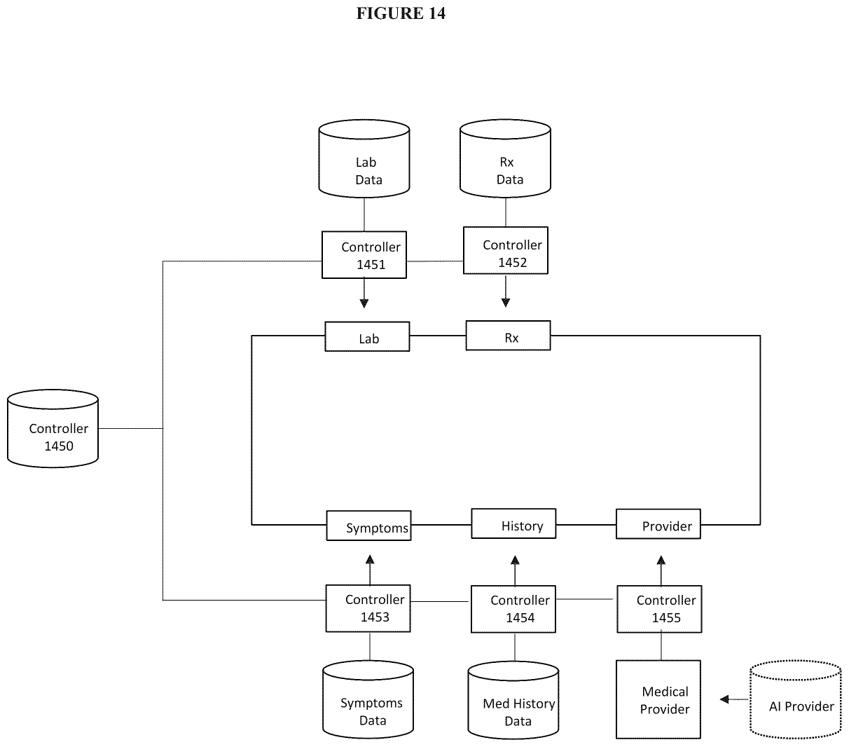

FIG. 14 illustrates a data flow for touch-free medical treatment, according to an aspect of the present disclosure.

As shown in FIG. 14, a variety of data is provided to a controller, and the controller provides the data directly or indirectly to a graphics processing unit (GPU) of the patient. The controller provides information based on lab data, prescription data, symptoms data, medical history data, and medical provider data. Some of the information provided to patients may be based on application of artificial intelligence as described herein. A service provider may guide the patient through treatment in an orderly communication flow using a controller, such as by using one or more computers (e.g., servers) that coordinate the collection of data, the application of algorithms, and the output of the data to user interfaces of electronic communications devices used by patients.

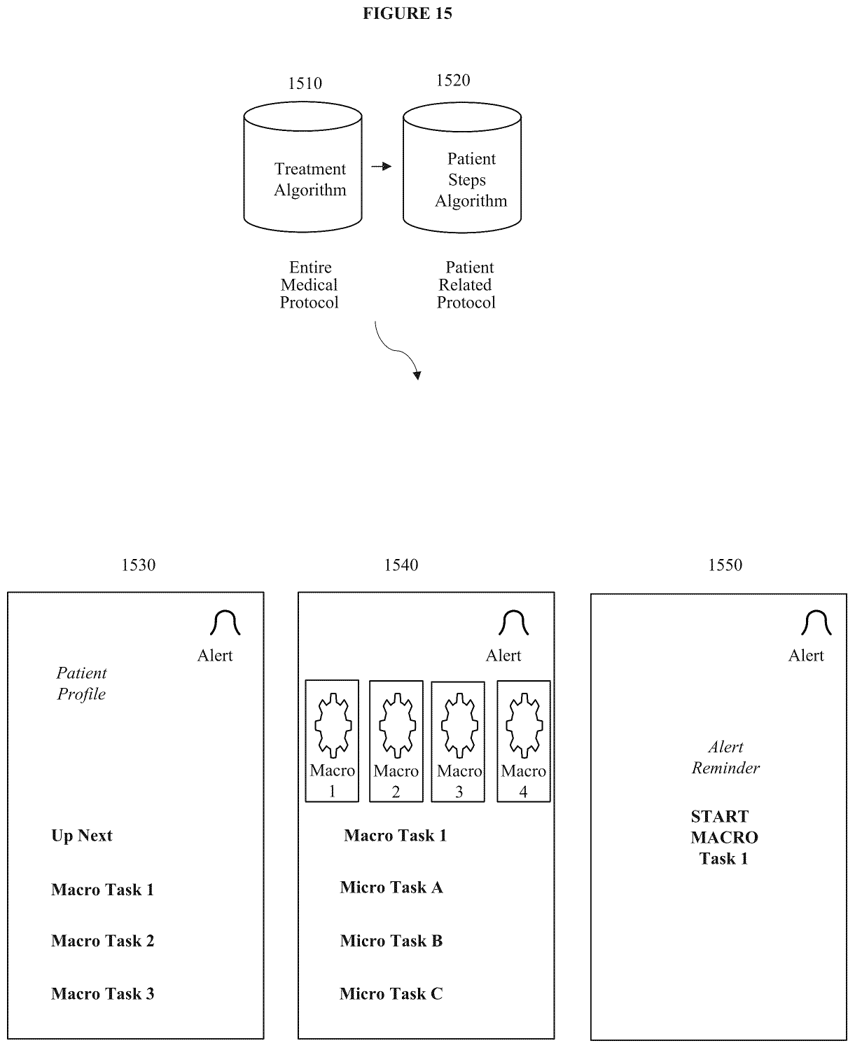

FIG. 15 illustrates a process flow for touch-free medical treatment, according to an aspect of the present disclosure.

In the process flow of FIG. 15, a treatment algorithm is applied at 1510, and a patient steps algorithm is applied at 1520. The treatment algorithm and the patient steps algorithm are applied by computers at the service provider.

Next, a guided treatment plan is provided to the patient by a series of user interfaces.

1530 shows a patient profile user interface that provides a patient with a series of macro tasks in order. Selection of each macro task may result in a different instance of the user interface 1540. For example, the user interface 1540 corresponds to the macro task 1, and may present a series of micro tasks to be completed in order. 1550 shows an alert reminder to remind the patient of the next macro task and/or micro task to complete.

An example of a macro tasks include obtaining lab data, and a corresponding micro task may be to complete the gathering of lab data. Another example of macro tasks is a pharmacy purchase, and a corresponding micro task may be to complete purchase of a prescription drug or dietary supplement.

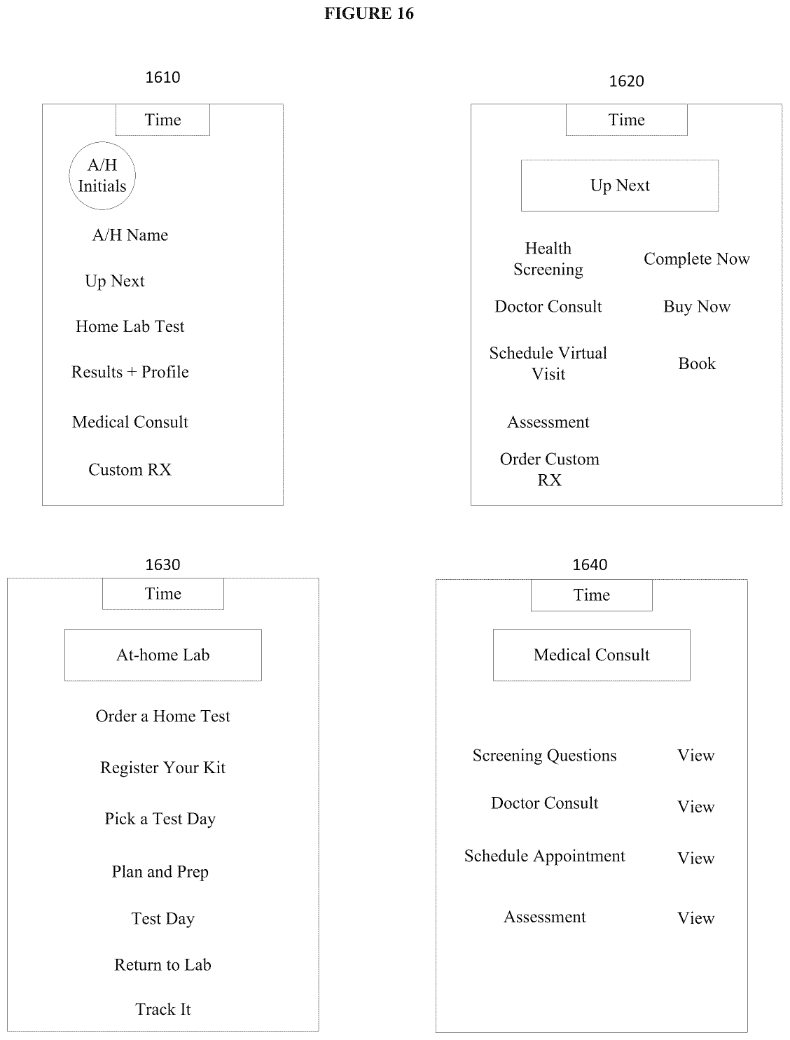

FIG. 16 illustrates a sequence of user interfaces for touch-free medical treatment, according to an aspect of the present disclosure.

The user interface 1610 shows a first user interface that is personalized for a patient, and that includes the patients name and a variety of selectable options such as up next, home lab test, results and profile, medical consult, and custom prescription options. The user interface 1620 shows a second user interface that indicates what is up next and includes a timeline along the left side to show what has been completed, such as an initial screening, a doctor consult, a scheduled virtual visit, an assessment, and a completion of a customized prescription. The user interface 1630 shows a third user interface with interactive instructions from ordering an at-home lab to tracking the results of the at-home lab after the at-home lab is returned to the service provider. The user interface 1640 shows a fourth user interface for a medical consult from screening questions to an assessment by a medical provider with the results of the medical consult.

Although only four user interfaces are shown in FIG. 16, it will be evident to one of ordinary skill in the appropriate art that more, and perhaps many more, user interfaces may be implemented via an application that patients can download onto local devices or interact with via a website.

As set forth above, a touchless medical treatment platform described herein enables a patient to take a lab test; enter risk factors, health history, and symptoms; undergo a clinical assessment with a healthcare provider; and obtain prescription(s) for the given condition, all from a private setting such as in their own home. Although touch-free medical treatment has been described with reference to several exemplary embodiments, it is understood that the words that have been used are words of description and illustration, rather than words of limitation. Changes may be made within the purview of the appended claims, as presently stated and as amended, without departing from the scope and spirit of touch-free medical treatment in its aspects. Although touch-free medical treatment has been described with reference to particular means, materials and embodiments, touch-free medical treatment is not intended to be limited to the particulars disclosed; rather touch-free medical treatment extends to all functionally equivalent structures, methods, and uses such as are within the scope of the appended claims.

While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a non-limiting, exemplary embodiment, the computer-readable medium can include a solid-state memory such as a memory card or other package that houses one or more non-volatile read-only memories. Further, the computer-readable medium can be a random-access memory or other volatile re-writable memory. Additionally, the computer-readable medium can include a magneto-optical or optical medium, such as a disk or tapes or other storage device to capture carrier wave signals such as a signal communicated over a transmission medium. Accordingly, the disclosure is considered to include any computer-readable medium or other equivalents and successor media, in which data or instructions may be stored.

The illustrations of the embodiments described herein are intended to provide a general understanding of the structure of the various embodiments. The illustrations are not intended to serve as a complete description of all the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for streamlining the disclosure. This disclosure is not to be interpreted as reflecting an intention that the claimed embodiments require more features than are expressly recited in each claim. Rather, as the following claims reflect, inventive subject matter may be directed to less than all the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to make or use the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. A method for touch-free medical treatment of a patient, comprising:
identifying a prospect as a candidate and providing the candidate with a lab test based on identifying the prospect as the candidate;
obtaining lab results from applying the lab test to a biometric sample from the candidate;
determining whether the candidate is suitable for treatment as a patient based on the lab results;
when the candidate is suitable for treatment as a patient, reviewing the lab results, candidate medical history, and demographic characteristics of the candidate in a review;
titrating a level of a customized drug or other therapy for the patient based on the review; and
prescribing the customized drug or other therapy for the patient at the titrated level based on the review.

2. The method of claim 1, wherein the treatment comprises hormone replacement therapy, and wherein the customized drug or other therapy is prescribed without face-to-face contact with a medical professional to obtain treatment via the customized drug or other therapy.

3. The method of claim 1, further comprising:
applying a trained artificial intelligence model to the lab results to determine whether the candidate is suitable for treatment as the patient.

4. The method of claim 3, wherein the trained artificial intelligence model is trained based on results of treating a plurality of additional patients treated before the patient.

5. The method of claim 1, further comprising:
applying a trained artificial intelligence model to data from reviewing the lab results to titrate the level of the customized drug or other therapy.

6. The method of claim 5, wherein the trained artificial intelligence model is trained based on results of customizing the customized drug or other therapy for a plurality of additional patients treated before the patient.

7. The method of claim 1, wherein the lab test is provided to the patient and the biometric sample is obtained from the patient without consultation with a doctor.

8. The method of claim 1, wherein the method is performed without a physical examination of the patient by a medical professional.

9. A tangible non-transitory computer readable storage medium that stores a computer program, the computer program, when executed by a processor, causing a computer apparatus to implement a process comprising:
identifying a prospect as a candidate and providing the candidate with a lab test based on identifying the prospect as the candidate;
obtaining lab results from applying the lab test to a biometric sample from the candidate;
determining whether the candidate is suitable for treatment as a patient based on the lab results;
when the candidate is suitable for treatment, reviewing the lab test, patient medical history, and demographic characteristics of the candidate in a review;
titrating a level of a customized drug or other therapy for the patient based on the review; and
prescribing the customized drug or other therapy for the patient at the titrated level based on the review.

10. The tangible non-transitory computer readable storage medium of claim 9, wherein the treatment comprises hormone replacement therapy, and wherein the customized drug or other therapy is prescribed without face-to-face contact with a medical professional to obtain treatment via the customized drug or other therapy.

11. The tangible non-transitory computer readable storage medium of claim 9, wherein the process implemented when the processor executes the computer program further comprises:
applying a trained artificial intelligence model to the lab results to determine whether the candidate is suitable for treatment as the patient.

12. The tangible non-transitory computer readable storage medium of claim 11, wherein the trained artificial intelligence model is trained based on results of treating a plurality of additional patients before the patient.

13. The tangible non-transitory computer readable storage medium of claim 12, wherein the trained artificial intelligence model is trained based on results of customizing the customized drug or other therapy for a plurality of additional patients treated before the patient.

14. The tangible non-transitory computer readable storage medium of claim 9, wherein the process implemented when the processor executes the computer program further comprises:
applying a trained artificial intelligence model to data from reviewing the lab results to titrate the level of the customized drug or other therapy.

15. The tangible non-transitory computer readable storage medium of claim 9, wherein the lab test is provided to the patient and the biometric sample is obtained from the patient without consultation with a doctor.

16. The tangible non-transitory computer readable storage medium of claim 9, wherein the process is performed without a physical examination of the patient by a medical professional.

17. A system for touch-free medical treatment of a patient, the system comprising:
a memory that stores instructions; and
a processor that executes the instructions, wherein, when executed by the processor, the instructions cause the system to:
apply an artificial intelligence model to patient data for the patient; and
identify treatment for disease states caused by menopause and perimenopause based on applying the artificial intelligence model to the patient data.
